(19) United States
(12) Patent Application Publication
(10) Pub. No.: US 2023/0277060 A1
(43) Pub. Date: Sep. 7, 2023

(54) Localizing and Imaging Magnetic Nanoparticles Assisted by Electron Paramagnetic Resonance

(71) Applicant: Mary Hitchcock Memorial Hospital, for itself and on behalf of Dartmouth-Hitchcock Clinic (D/B/A “Dar, Lebanon, NH (US)

(72) Inventor: John B. Weaver, Hanover, NH (US)

(21) Appl. No.: 18/116,742

(22) Filed: Mar. 2, 2023

Related U.S. Application Data
(60) Provisional application No. 63/315,626, filed on Mar. 2, 2022.

(51) Int. Cl. A61B 5/00; A61B 5/055; A61N 2/00; G01R 33/60

(52) U.S. Cl. CPC A61B 5/0036; A61B 5/055; A61N 2/004; G01R 33/60; B82Y 25/00

(57) ABSTRACT

An MNP machine provides a magnetic bias field to a sample space; drive coils bracketing the sample space; pickup coils coupled through amplifiers to a computer; and a radio frequency (RF) stimulus coil driven at an electron paramagnetic resonance (EPR) frequency of MNPs in the bias field. The computer is configured to provide a MNP Brownian motion spectrum from the signals or magnetic particle images. A method of imaging MNP concentrations in a subject includes applying a magnetic bias field having a gradient; applying RF at an EPR frequency of the MNPs in the magnetic bias field; sweeping either magnetic bias field strength or radio frequency to sweep a surface of resonance through the subject; observing EPR resonances of the MNPs; rotating the magnetic bias field relative to the subject; repeating sweeping the surface of resonance through the subject; and reconstructing a three-dimensional model of MNP concentrations of the subject.

### CLAIM TO PRIORITY

The present application claims priority to U.S. Provisional patent application 63/315,626 filed 2 Mar. 2022. The entire contents of the aforementioned provisional patent application are incorporated herein by reference.

### BACKGROUND

Electron paramagnetic resonance (EPR), also known as electron spin resonance (ESR), is a phenomenon where unpaired electrons in materials resonate at specific frequencies (the Larmour frequency) in a static bias magnetic field. This resonance resembles, but is at very different frequencies from, the nuclear magnetic resonance (NMR) of unpaired protons (typically hydrogen protons) commonly taken advantage of in NMR spectrographs used for chemical analysis and in magnetic resonance imaging (MRI) as used for medical imaging. EPR Larmor frequencies also increase with magnetic field strength.

EPR resonances involve interactions of electrons with an electromagnetic wave and occur at a frequency dependent on the magnetic field present at the resonating electron.

Magnetic nanoparticles (MNPs) are nanoparticles of size 1-10³ nanometers diameter, and magnetic microbeads of size 10³-10⁶ nanometers diameter, which incorporate a magnetic core including at least one magnetic material. MNPs are of interest in biology and medicine because research has shown MNPs can be tagged or labeled with tissue-selective agents such as antibodies and other ligands. Concentrations of MNPs in tissue can be imaged or localized with several techniques thereby identifying tissues binding tissue-selective labeled MNPs. MNPs can be administered through catheters to specific tissue locations thereby forming MNP concentrations in those tissues. Further, MNP concentrations in tissue can be electromagnetically heated to destroy tissue containing the MNPs. MNPs can also be tagged with antineoplastic agents and magnetically guided to specific tumor locations, and for other purposes. Localizing concentrations of MNPs in tissue is known as MNP imaging (MPI), MPI has been demonstrated with prototype machines.

Since signals differ between bound and free MNPs, it is possible to distinguish concentrations of bound MNPs, such as MNPs bound in blood clots in vivo, or labeled MNPs bound to a ligand in vitro, from unbound MNPs using magnetic particle spectroscopy (MPS) a process termed magnetic spectroscopy of Brownian motion (MSB). It is therefore believed that there is a large potential market for improved methods and devices for localizing, and identifying bound states of, MNPs in human and other biological tissues.

Historically, EPR phenomena have not been used in equipment used for imaging and localizing concentrations of MNPs in tissue.

The signal detected in prior magnetic particle imaging (MPI) and magnetic particle spectroscopy (MPS) is generated by a change in direction of the magnetization of the magnetic particles.

### SUMMARY

A magnetic nanoparticle (MNP) machine has magnets providing a bias field to a sample space; a pair of resonant drive coils bracketing the sample space;
at least one pickup coil coupled to a lock-in amplifier, the lock-in amplifier coupled to provide signals to a computer; and a radio frequency (RF) stimulus coil driven at an electron paramagnetic resonance (EPR) frequency of MNPs in the bias field where the computer is configured to provide a MNP Brownian motion spectrum from the signals magnetic particle images from signals received from the lock-in amplifier.

A method of imaging first magnetic nanoparticle (MNP) concentrations in a subject includes applying a magnetic bias field having a gradient to the subject; applying pulses of a radio frequency field to the subject at an electron paramagnetic resonant frequency of the first MNPs in the magnetic bias field; sweeping a parameter selected from the group consisting of strength of the magnetic bias field strength and the radio frequency to sweep a surface of resonance through the subject; observing electron paramagnetic resonances of the first MNPs; rotating the magnetic bias field relative to the subject and sweeping the surface of resonance through the subject while observing additional electron paramagnetic resonances of the first MNPs; and reconstructing first MNP concentrations in a first three-dimensional model of the subject.

### BRIEF DESCRIPTION OF THE FIGURES

FIG. 1 illustrates sense signals resulting when a magnetic field is swept through resonance with an applied electromagnetic field.

FIG. 2 is a schematic diagram of a MNP spectroscopy machine adapted to apply a radio frequency electromagnetic field to stimulate EPR resonance in nanoparticles.

FIG. 3A is a schematic diagram of a MNP imaging (MPI) machine adapted to use a radio frequency electromagnetic field to stimulate EPR resonance in nanoparticles.

FIG. 3B is a schematic diagram of an alternative MNP imaging machine.

FIG. 4A is a simulation graph of nanoparticle magnetization of MNPs in an MPI machine where MNPs are located near a peak magnetic field of a magnetic field gradient, and FIG. 4B is a simulation graph of detectable signals produced by the MNPs of FIG. 4A.

FIG. 4C is a simulation graph of nanoparticle magnetization of MNPs in an MPI machine where MNPs are located centrally in a magnetic field of a magnetic field gradient, and FIG. 4D is a simulation graph of detectable signals produced by the MNPs of FIG. 4C.

FIG. 5A illustrates a machine for heat-treating abnormal tissue in a subject where heating of the subject is localized to a surface within the subject where MNPs in an applied magnetic field have Larmor resonance with an applied RF field with mechanical rotation.

FIG. 5B illustrates a machine for heat-treating abnormal tissue in a subject where heating of the subject is localized to a surface within the subject where MNPs in an applied magnetic field with electronic rotation have Larmor resonance with an applied RF field.

FIG. 6 is a schematic diagram of localizing resonance with an endoscopic magnet.

FIG. 7 is a schematic diagram of an alternative magnetic particle imaging machine with axial main magnets and trim magnets for generating and warping magnetic field gradients, an RF stimulation coil, and sense coils.

FIG. 8 is as flowchart of a method of imaging magnetic nanoparticles using the machine herein described.

FIG. 9 is a flowchart of a method of generating an enhanced image of targeted magnetic nanoparticles in a subject by imaging targeted magnetic nanoparticles, imaging nontargeted nanoparticles, and subtracting the nontargeted image from the targeted image.

### DETAILED DESCRIPTION OF THE EMBODIMENTS

When unpaired electrons of a magnetic nanoparticle (MNP) are subjected to an applied magnetic field, they tend to align with, or align against, that magnetic field; as time progresses, they align with the applied magnetic field.

When unpaired electrons of a MNP having magnetic domains aligned with or against a magnetic field interact through EPR resonances with an applied electromagnetic energy, the up and down spin states of those electrons is either 1) reversed in some MNPs or, 2) for higher power, equalized thereby eliminating the magnetization of the MNPs.

When the magnetization of the MNPs is eliminated by the EPR resonance, upon termination of the applied electromagnetic energy the MNPs once again align with, or align against, that magnetic field; as time progresses, they align with the magnetic field.

The realignment of MNPs with the magnetic field gives a detectable signal that varies in magnitude with quantity of the electrons realigning with the magnetic field and a time derivative of the changing magnetization.

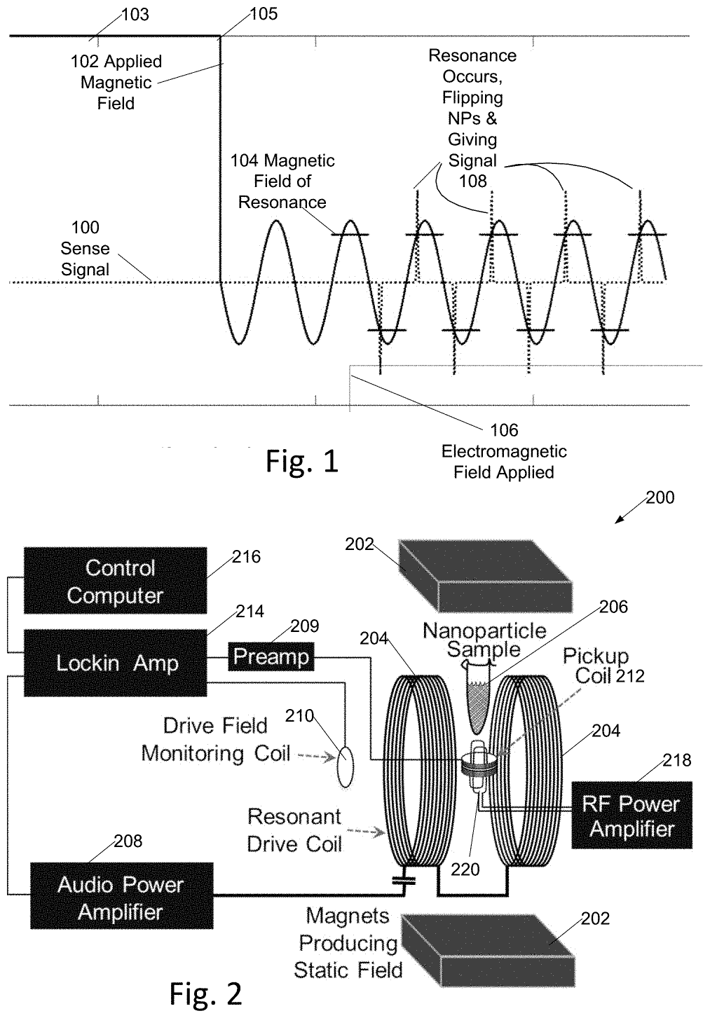

In an embodiment, a magnetic field 102 (FIG. 1) is applied to materials, which may include biological materials or a human, which contain MNPs. Initially, at time 103, this magnetic field is a high static bias field to which spins of the MNPs align. At time 105, this magnetic field is reduced in intensity and begins alternating, as the magnetic field alternates it passes through a magnetic field intensity 104 at which EPR resonance occurs with a particular radio frequency (RF) of electromagnetic radiation that is applied at time 106. When that particular frequency of electromagnetic radiation is applied and the magnetic field strength passes through the magnetic field intensity 104 at which EPR resonance occurs, resonance causes the aligned spins to flip or randomize as resonance ceases these spins realign and give a detectable signal 108 in a pickup coil. Without resonance, sense signals 100 are flat. Where a biasing magnetic field gradient exists, the resonance will occur only when total magnetic field at the MNPs is such that the electron spins of the MNPs resonate at the particular applied frequency; by determining when this resonance occurs, we can determine where in the magnetic field gradient the MNPs are located. In principle, either the magnetic field intensity or the frequency of the applied electromagnetic field may be swept through resonance to localize MNP concentrations in the biasing magnetic field gradient. With each direction of magnetic field gradient, as field strength or frequency is swept resonance of MNPs occurs along a surface in a three-dimensional imaging space; by changing direction of the magnetic gradient we change an angle of a surface of resonance and further localize the MNP concentrations as being at intersections of surfaces of resonance; by using a sequence of three or more noncoplanar magnetic field gradient directions with swept magnetic fields or frequencies we can localize and quantify MNP concentrations to points in a three-dimensional imaging space.

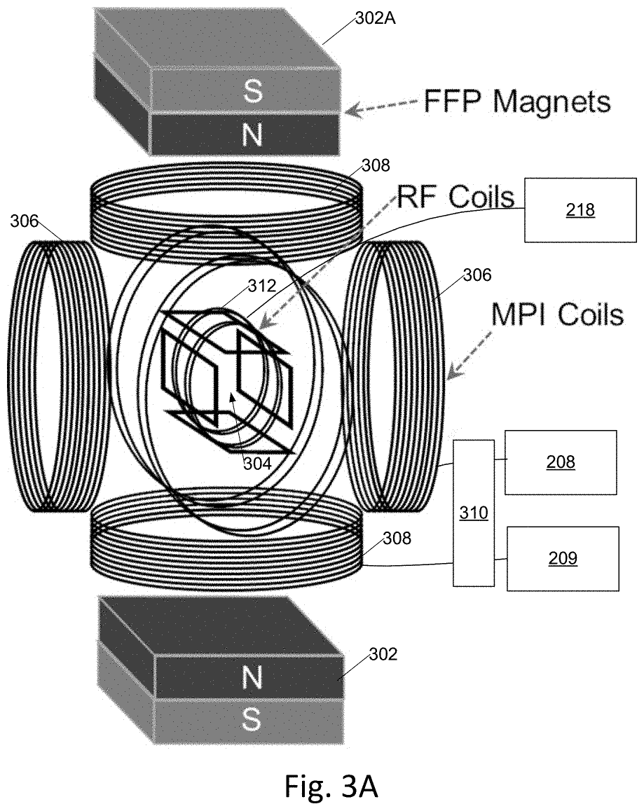

In a MNP spectroscopy machine 200 (FIG. 2) adapted to apply a radio frequency electromagnetic field to MNPs in a biasing magnetic field, magnets 202 provide a bias field to a sample 206; magnets 202 are magnetically coupled together by an iron core (not shown). A pair of resonant drive coils 204 bracket sample 206, the resonant drive coils 204 are driven by an audio amplifier 208. The drive magnetic field provided by the resonant drive coils 204 is sensed by a drive monitoring coil 210 and at least one, and preferably paired, pickup coils 212 oriented at right angles to the resonant drive coils 204 provides responses of MNPs in sample 206 through a preamplifier to a lock-in amplifier 214. Signals from lock-in amplifier 214 are digitized and provided to a computer 216 configured to provide a MNP Brownian motion spectrum from the signals. Computer 216 also controls operation of the system, including RF sources, audio sources, magnets, and resonant drive coils 204. A radio frequency signal is provided by an RF amplifier 218 at an EPR resonant frequency. In this embodiment, an audio frequency applied field generated by the resonant drive coils 204 and the RF signal is applied through RF stimulus coil 220 by RF amplifier 218. The electrons forming the MNP magnetization will reach resonance when the total magnetic field at the MNP times the Gyromagnetic ratio is equal to the applied RF field frequency. The total field is the combination of the audio magnetic field, the static magnetic field and the field formed by the crystal structure of the magnetic core of the MNPs.

Static magnetic fields as described herein are produced by magnets that may be permanent magnets, electromagnet coils, or combinations of permanent magnets and electromagnet coils. Where magnetic fields are disclosed as have gradients they are also produced by magnets that may be permanent magnets, electromagnet coils, or combinations of permanent magnets and electromagnet coils. Where magnetic fields are disclosed as being swept in intensity these fields may be produced by either electromagnet coils or by a combination of permanent magnets and electromagnet coils.

When the electrons resonate, MNP magnetization alignment is disturbed in sample 206 and a signal is picked up by pickup coils 212. Timing of the signal relative to the audio signal waveform, as analyzed by computer 216, provides a MNP spectrum by identifying the time lag before the MNPs reach resonance.

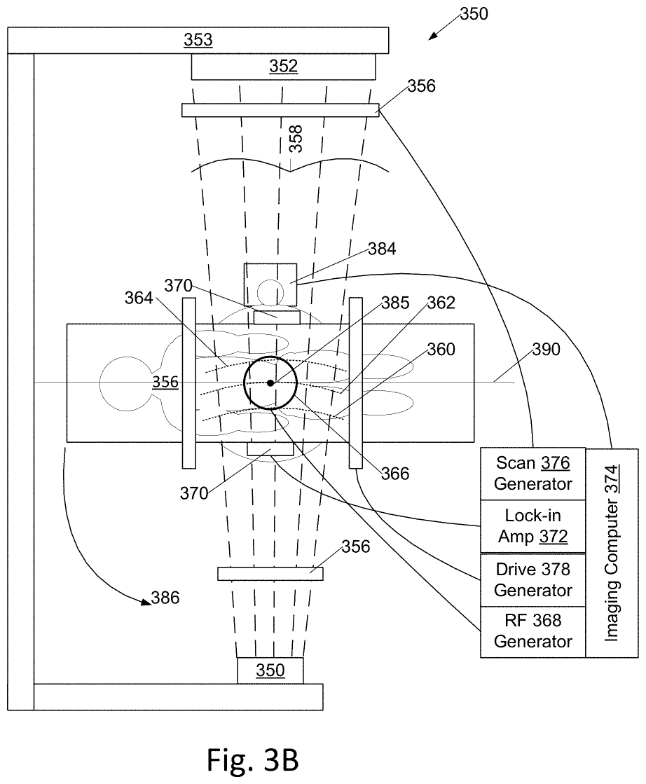

In a MNP imaging (MPI) embodiment 300 (FIG. 3A), opposed permanent magnets 302 (or equivalent coils) provide a magnetic field with a set of magnetic field gradients with a “field free point” (FFP) that can be moved using the “MPI coils” 306, 308, 309 across a space 304 within which a subject or biological sample (not shown in FIG. 3A) may be positioned. The current in the MPI coils (306, 308, 309) is driven by an audio amplifier 208. As the FFP passes over MNPs, the MNPs flip orientation producing a signal in pickup coils 312 (only one of the three pairs of coils are shown in FIG. 3A) amplified by a preamp and analog to digital converter 209. Alternatively, the “MPI coils” 306, 308, 309 can be used to both drive the FFP point and detect the signal using a switch 310 to direct the signal to the preamp 209. Space 304 is also surrounded by RF coils 312 coupled to be driven by RF amplifier 218. Again to keep the figure readable, only one of three pairs of RF coils are shown. The combination of RF coils excited determine a direction of the RF field produced. The orientation of the fields producing the FFP (302 and 308, 309, 309) determine the direction of the applied field. The MNPs located where the fields producing the FFP and the RF fields produce a resonance will absorb RF and flip orientation, in some embodiments this is on a boxlike surface of low field surrounding the FFP. Additional electronics similar to that of FIG. 2 is provided but not shown in FIG. 3A for simplicity, including lock-in amplifier 214 or other amplifier/analog to digital conversion unit coupled to computer 216. The computer 216 is configured to perform magnetic particle imaging based on the signals it receives from the lock-in amplifier. Operation is similar to the embodiment of FIG. 2 in that timing of the signal relative to the audio signal waveform, as analyzed by computer 216, is determined, this timing however provides not a spectrum but a surface on which the MNPs are located within the subject or biological sample in space 304. Additional imaging may be obtained by switching operation of the drive and sensing coils by switching switch 310.

Imaging in the embodiment of FIG. 3A is performed by using the MPI coils 306, 308, 309 as electromagnets on the main magnets 302 to scan the FFP, including the surface of low field surrounding the FFP, through space 304 and across any subject bearing MNPs while providing an audio-frequency AC field in the drive coils on top of the main field, and recording and analyzing signals received by the sense coils as the FFP is scanned, to identify locations of the MNPs. In alternative embodiments, sense coils may be supplemented by other forms of magnetic field sensing devices or magnetometers, such as but not limited to Hall-effect sensors, superconducting quantum interference devices (SQUIDs), and magneto-optic Kerr-effect magnetometers.

An alternative embodiment of a machine 350 (FIG. 3B) to image MNP concentrations uses main magnets 352, 354 that are aligned, not opposed, to create a magnetic field 358 having a magnetic field gradient across an imaging space within which a subject 356, or portion of a subject, may be positioned. A magnetic core 353 couples magnets 352, 354.

Either the main magnets 352, 354 are electromagnets, or scanning electromagnets 356, are provided that can alter intensity of the magnetic field 358 thereby moving a surface (dashed arcs) of resonance from a first location 360 to other locations 362, 364 through a region of interest in the imaging space. At least one pair of RF coils 366 are provided near the region of interest and are driven by an RF generator 368 to excite resonance of MNPs within the surface of resonance 360. Sense coils 370 are provided to receive signals from MNPs as they realign with magnetic field 358. Sense coils are coupled through a lock-in amplifier 372 or other amplifier/analog-to-digital converter to provide sense signals to an imaging computer 374 that controls magnets and RF sources plus analyzes sense signals to determine when EPR resonance disturbs alignment of MNPs with magnetic field 358. Computer 374 also controls RF generator 368 and a scan generator 376 that drives scanning electromagnets 358 or main magnets 352, 354 to move the surface of resonance. In a particular embodiment, computer 374 may also control an audio drive generator 378, coupled to drive coils 380.

Operation of the embodiment of FIG. 3B is similar to the embodiment of FIG. 2 in that timing of the signal relative to the audio signal waveform, as analyzed by computer 374, is determined; this timing however provides not a spectrum but surfaces on which the MNPs are located within the subject or biological sample in the region of interest within the imaging space.

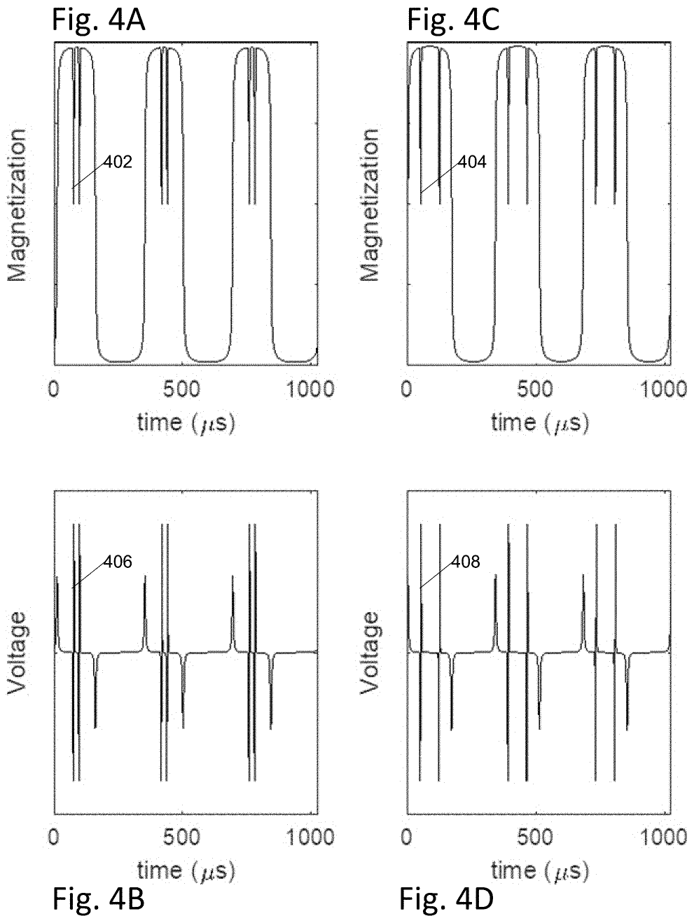

Imaging in the system of FIG. 3B results because when magnetic field 358 having a spatial intensity gradient is swept in strength across a subject or biological specimen while a constant RF electromagnetic field is applied, MNPs in the subject or specimen resonate with the constant RF essentially only along a surface where the magnetic field induces a Larmor frequency in the MNPs that matches the RF frequency. For example, if the nanoparticles are located where the magnetic field reaches resonance only late in the sweep, the nanoparticle magnetization is that of FIG. 4A and detectable signals produced by the MNPs are of FIG. 4B. If the MNPs are located where the magnetic field reaches resonance earlier in the sweep, the nanoparticle magnetization is that of FIG. 4C and detectable signals are those of FIG. 4D. The dips 402, 404 in nanoparticle magnetization and peaks 406, 408 in detectable signals are because the resonance disrupts alignment of the MNPs, and they must realign as the resonance passes them.

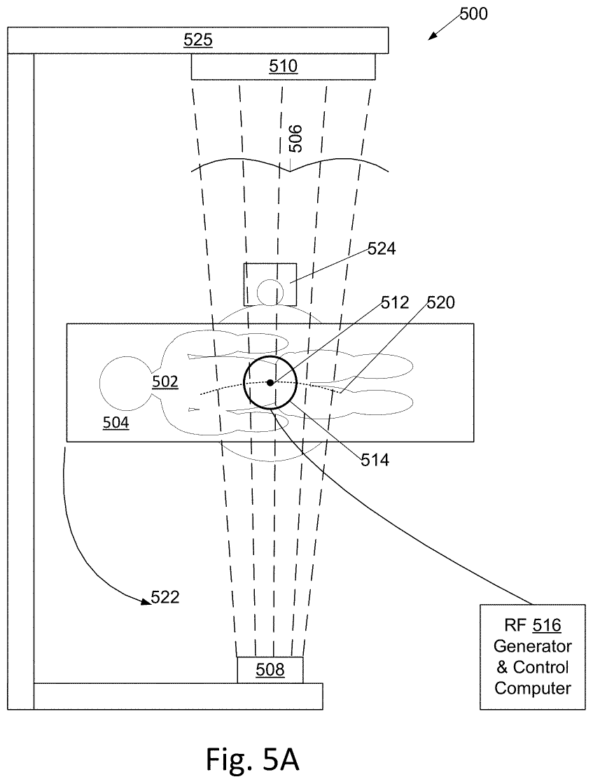

In the embodiment of FIG. 3B, after determining surfaces within the subject or biological sample having resonating MNPs, either the subject or biological sample is rotated relative to the magnetic field and/or the magnetic field is rotated by, for example, deactivating main magnets 352, 354 and activating another set of main magnets (not shown in FIG. 3B for simplicity but similar to those of the MNP hyperthermia treatment machine of FIG. 5B) to provide a rotated magnetic field, by using a mechanical rotator 384 about an axis 385 operating under control of computer 374, or both—in a particular embodiment multiple sets of main magnets are provided to provide surfaces of resonance at differing angles from vertical about a horizontal axis 390 through the region of interest while a rotator provides surfaces at different angles of rotation about the vertical axis 385. Additional surfaces are determined within the subject or biological sample where resonating MNPs are located at multiple angles of rotation 386. After multiple surfaces are determined each at a different rotation both about the vertical 385 axis and about a horizontal axis 390, computer 374 analyzes the determined surfaces where resonating MNPs are located and reconstructs locations of the resonating MNPs in a voxel-based three-dimensional model of the subject or biological sample by fitting concentration parameters of a the three-dimensional model to signal strengths of observed surfaces in a manner similar to MRI reconstruction.

In an alternative embodiment resembling that of FIG. 3B, the bias magnetic field, including the magnetic field gradients, is provided by electromagnets of an MRI machine such as commonly used to image concentrations of unpaired hydrogen protons, although the electromagnets may be operated significantly different—usually lower—currents for EPR imaging of magnetic nanoparticles than for MRI imaging. Since EPR resonance occurs at a frequency that increases with magnetic field strength, and for the same magnetic field strength at far higher frequencies than the hydrogen nuclear magnetic resonances imaged by MRI machines in normal operation, the MRI machine bias field magnets are operated at far lower currents than during MRI operation. By reducing the MRI machine bias field strength the RF frequency of resonance is low enough to penetrate a subject or biological material containing MNPs and disposed within the MRI machine. In an alternative embodiment, where the MRI machine is a low-field permanent magnet MRI machine, an additional pair of coils are provided and pulsed with a high current pulse to oppose fields provided by the bias magnets of the MRI machine and reduce field strength sufficiently that the RF frequency is low enough to penetrate a subject or biological material containing MNPs and disposed within the MRI machine.

We note that the EPR resonant frequency of MNPs depends in part on composition of the MNPs, and that MNPs may be made with iron oxide cores, with a ferrite core, with metallic iron cores, with a cobalt-containing magnetic core, with an iron-platinum alloy core, or with cores of other magnetic alloys. The magnetic cores can be either uniform of a single magnetic material or in “core-shell” or “sandwich” geometries with two magnetic materials. The two materials can be of different permeability allowing one material to flip magnetizations more or less easily.

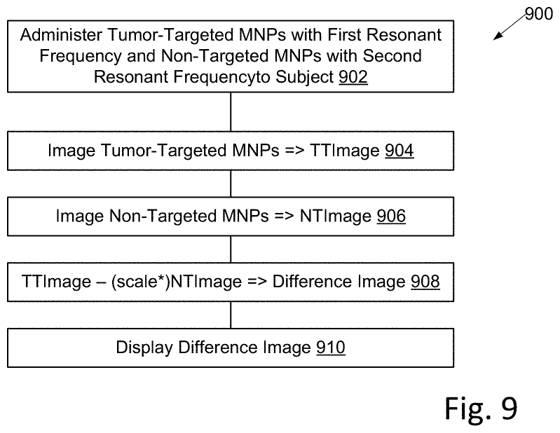

It is therefore possible to form a duplex contrast agent comprising a first and a second MNPs where the first MNPs are labeled with a first ligand or antibody and the second MNPs are either unlabeled or labeled with a second ligand or antibody. In an embodiment of enhanced MNP imaging 900, after administration of this duplex contrast agent either as a single duplex agent or as separate injections to a subject 902 (FIG. 9) biological sample, we propose imaging the first MNPs 904 with a first applied EPR frequency to form a first three-dimensional model TTImage of the subject or biological sample and imaging the second MNPs 906 with a second EPR frequency differing from the first EPR frequency (or alternatively with a second base magnetic field strength) to form a second three-dimensional model NTImage of the subject or biological sample. We can display the first and second three-dimensional models separately to a user or can scale as necessary and subtract 908 the second three-dimensional model NTImage from the first three-dimensional model TTI mage to form a difference three-dimensional model and display 910 the difference three-dimensional model to the user, display may be done in tomographic slices as known in three dimensional imaging. When first MNPs are targeted to tumor with a first ligand, antibody, or other selective agent so first MNPs concentrate in tumor to a greater extent than in normal tissue, while second MNPs are nontargeted, the difference three-dimensional model cancels background first MNPs and thus increases contrast of tumor over normal tissue.

In another embodiment the EPR resonance is taken advantage of during RF-heating of MNPs located within a tumor or other abnormal tissue that is to be heat-treated. It is known that application of high temperatures to tumor tissue can destroy that tumor tissue, and that while heating tumor tissue it is desirable to minimize heat applied to nearby normal tissue. The machine of FIG. 3A can be used to heat MNPs instead of, or in addition to, imaging them. When the electrons on the MNPs are at resonance, the electrons absorb the RF energy and, if the RF energy is intense enough, the temperature of tissue around the MNPs will increase allowing either hyperthermia or ablation of the tissue. The location heated can be controlled by selecting the fields producing the FFP to produce resonance along selected surfaces locations and no other locations. The increased absorption of RF energy at EPR resonance allows the RF field to be low enough energy to penetrate other tissue of the subject without absorption.

In a machine 500 (FIG. 5A) for heat-treating abnormal tissue in a subject 502 or other biological material, the subject or biological material is placed on a rotatable table 504 in a treatment space having a magnetic field 506 with an intensity gradient, the field being produced by magnets 508, 510 that may be coupled by a magnetic core 525. The rotatable table 504 is rotatable about an axis 512, and an RF heating coil 514 is positioned over the abnormal tissue (not shown for simplicity) in subject 502. When RF power is provided by a RF generator and control computer 516 to RF heating coil 514, EPR resonance occurs in MNPs within subject 502 or biological material along a surface 520 determined by the magnetic field strength and the magnetic field gradient; in a particular embodiment the magnetic field is configured so surface 520 intersects the axis 512, and the subject 502 is positioned so the abnormal tissue is also located at the axis 512. While the subject 502 to the RF field produced by RF heating coil 514, the subject may be rotated 522 about axis 512 by a rotator motor 524 to reduce heating concentrations in normal tissue while continually heating abnormal tissue positioned at axis 512. Where necessary, the volume to be heat treated about the axis 512 may be increased by modulating magnetic field strength.

In an alternative embodiment 550, FIG. 5B, instead of mechanically rotating the subject 502 relative to the magnetic field, the magnetic field is effectively rotated relative to the subject while RF heating coil 514 is energized by an RF generator and control computer (not shown in FIG. 5B for simplicity). This can be done by providing multiple sets of magnets, such as magnet pair 552, 554, magnet pair 556, 558, magnet pair 560, 562, and magnet pair 564, 566, and operating the magnet pairs sequentially. For example, magnet pair 552, 554 may be operated to cause heating along surface 568, then magnet pair 552, 554 is turned off and magnet pair 556, 568 is operated to cause heating along surface 570, then magnet pair 556, 568 is turned off and magnet pair 560, 562 is operated to cause resonance and heating along surface 572, and so on; after all magnet pairs are operated the cycle repeats. by operating magnet pairs simultaneously even more angles of magnetic field gradient can be obtained. Thermal inertia of tissue allows cumulative heating of abnormal tissue at the intersection of surfaces 570, 572, 568 to effective hyperthermia treatment levels, while tissue elsewhere remains at lower temperatures even in a single resonance surface 572.

In an alternative embodiment, a configuration similar to that of FIG. 5A has both at least one pair of imaging RF coils like imaging coils 366 is provided where RF heating coil 514 is relatively smaller than the imaging coils. In this embodiment, imaging may be performed as previously described using the imaging coils prior to, or during brief pauses during, heating using RF heating coil 514. The small size of heating coil 514 permits focusing heating on particular points within a subject, such as within a tumor—in a particular embodiment, heating coil 514 is on an endoscope or catheter to enable positioning within or directly adjacent to the tumor. Further, the imaging coils used during brief pauses during heating may be used to observe MNP resonance spectra of Brownian motion to determine, and map in a 3-D voxel-based model, temperature of MNPs throughout a region of interest in the subject and thereby monitor hyperthermal treatment of abnormal tissue.

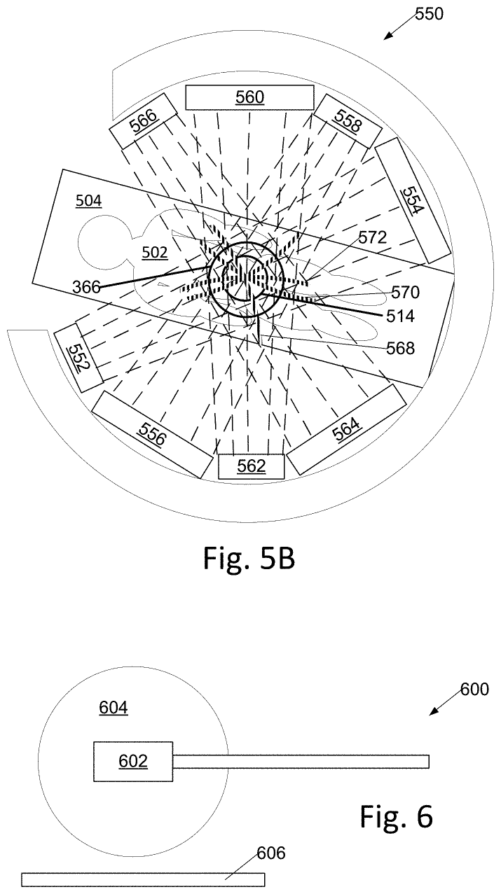

In an alternative embodiment 600 (FIG. 6), an endoscope, ureteroscope, or similar device is fitted with a magnet 602, magnet 602 generates a localized, nonuniform, magnetic field within abnormal tissue 604. When RF energy is applied through a coil 606 at a Larmor frequency of some of the MNPs in abnormal tissue, localized heating of the abnormal tissue 604 occurs.

In a particular embodiment, the MNPs are complexed with antibodies to a specific abnormal tissue type, such as a tumor, such that the MNPs concentrate in tissue of that specific abnormal tissue type. In this embodiment, the RF energy preferentially heats tissue the specific abnormal tissue type within which the MNPs concentrate.

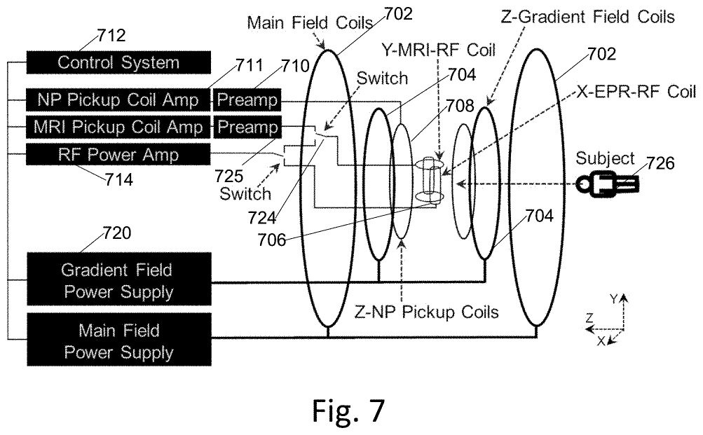

In an alternative magnetic particle imaging machine 700 configured to also perform nuclear magnetic resonance imaging (MRI), there are axial main magnets 702, used for producing the main magnetic field for MRI machine operated at normal strength for MRI imaging and at very low field strength to produce EPR resonance instead of nuclear magnetic resonance for magnetic particle imaging. Machine 700 has gradient magnets 704 (only Z axis gradient magnets 704 are shown, there are also perpendicular gradient magnets oriented in the X and Y axes that are omitted from FIG. 7 for clarity) for generating magnetic field gradients in differing directions. The gradient magnets are driven by gradient magnet drivers 720. Nanoparticle sense coils 708 are coupled through a preamp 710 and lock-in amp 711 or another analog-to-digital amplification system to an imaging computer and control system 712. RF coils 706 are positioned near an imaging space within which a subject 726 may be positioned, the RF coils 706 are driven by a pulsed RF generator 714 to produce EPR resonance in magnetic nanoparticles within subject 726 when machine 700 is in magnetic nanoparticle imaging mode. The axial main magnets 702, with trim and gradient magnets 704, generate magnetic gradients where unpaired electrons of magnetic nanoparticles will resonate along surfaces (not shown) at a variety of angles (not shown for simplicity) relative to a subject 726 that may be scanned through subject 726 by altering field strengths of main and/or gradient fields. This effectively allows electronic steering of the surfaces for detection of magnetic nanoparticle heating or magnetic particle imaging as herein described by detecting realignment of magnetic nanoparticles with the sense coils 708. In a particular embodiment, RF coils 706 are operated through a switch 724 in MRI imaging mode and coupled as MRI sense coils to MRI amplification and sense circuitry 725. In an embodiment, composite images are generated with MRI images in a first color and magnetic nanoparticle location data in a second color.

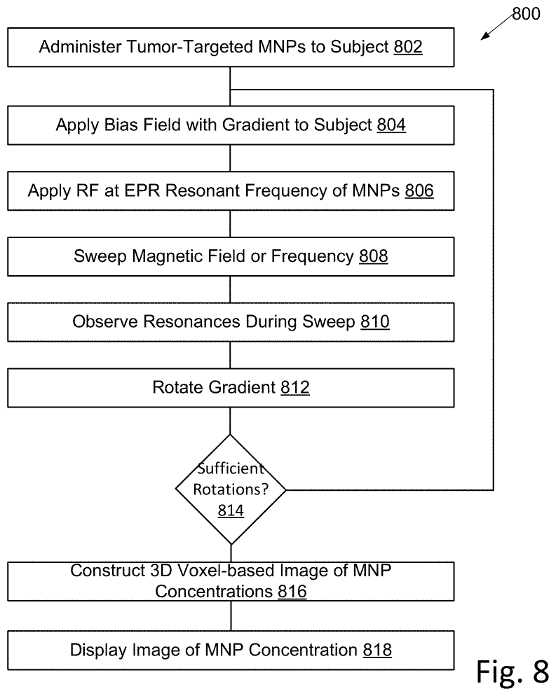

To review, a method of imaging 800 magnetic nanoparticle concentrations in a subject (FIG. 8) operates by administering MNPs 802 to the subject and allowing them to reach desired organs. A magnetic bias field is then applied 804 to the subject, and pulses of a radio frequency field are applied 806 to the subject at an electron paramagnetic resonant frequency of MNPs in the magnetic bias field forming a surface of resonance within the subject. Either the magnetic bias field strength or the radio frequency is swept 808 through the subject while resonances are observed 810 with appropriate sense coils and electronics. The magnetic bias field is then rotated 812 relative to the subject. Steps of applying pulses of the radio frequency field 806 forming a surface of resonance within the subject, sweeping 808 the magnetic field strength or sweeping the radio frequency while observing resonances 810, and rotating the magnetic field gradient 812 are repeated 814 until sufficient data has been obtained. A computer then sets up a voxel-based model of the subject and uses the observations of resonances to reconstruct MNP concentrations at each voxel within the model. The MNP concentrations are 3D images that may then be displayed 818 as tomographic slices.

In particular embodiments, the MNPs are tagged with antibodies specific for a specific tumor type, and the 3D images represent tumor locations within the subject. In another embodiment, the MNPs are tagged with a ligand capable of binding to a particular tissue type, and the 3D images represent distribution of that tissue type within the subject.

A significant difference between the present system and many other magnetic nanoparticle imaging systems is that there is no “field free point” in the sample space because presence of a magnetic field at a magnetic nanoparticle is necessary for electron paramagnetic resonances to occur.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. It is also anticipated that steps of methods may be performed in an order different from that illustrated and still be within the meaning of the claims that follow.

What is claimed is:

1. A magnetic nanoparticle (MNP) electron paramagnetic resonance machine comprising:
magnets configured to provide a magnetic field to a sample space;
at least one pickup coil or magnetometer configured to detect magnetization in the sample space and coupled to provide magnetic data a computer; and
at least one RF driver and RF field coil configured to generate an RF field at a Larmor resonance frequency of unpaired electrons in MNPs that may be present in the sample space;
where the computer is configured to control the magnets and RF driver and produce either resonance spectra from the MNPs or images of MNP concentrations from the magnetic data.

2. The machine of claim 1 where the magnetic field is oriented along a magnetic field axis is an alternating field and the pickup coil is oriented along the magnetic field axis and the RF field is oriented perpendicular to that the magnetic field axis; and
where the RF field is at the EPR frequency of the unpaired electrons of the MNPs at intervals during cycles of the alternating field.

3. The machine of claim 1 where the magnetic field comprises a static field oriented along a static magnetic field axis and the pickup coil is oriented along the static magnetic field axis; the magnetic field further comprises an alternating magnetic field oriented along an alternating field axis perpendicular to the static magnetic field axis and the RF coil is oriented along an RF axis perpendicular to the alternating field axis and the static magnetitic field axis;
where the RF field is at the EPR frequency of the unpaired electrons of the MNPs at intervals during cycles of the alternating field.

4. The machine of claim 1 where the static magnetic and magnetometer are oriented along a magnetic field axis and the RF field is along an axis perpendicular to the magnetic field axis.

5. The machine of claim 1 where the magnets configured to provide a magnetic field to the sample space and an RF Driver and MRI RF coils are configured to provide an RF field at resonant frequency of hydrogen protons to make MR images of hydrogen.

6. An MNP heat-treatment machine comprising the MNP machine of claim 1 wherein at least one RF coil is driven with sufficient power to heat MNPs in the sample space at a frequency that is the EPR Larmor frequency of unpaired electrons in the MNPs.

7. The MNP heat-treatment machine of claim 6 wherein the at least one RF coil driven with sufficient power to heat MNPs in the sample space is an unpaired drive coil smaller than the at least one pair of drive coils.

8. The MNP heat treatment machine of claim 7 wherein the computer is adapted to determine MNP Brownian motion spectra to monitor temperature of MNPs during pauses of MNP heating.

9. The MNP heat treatment machine of claim 8 wherein the computer is adapted to map temperature through the sample space from the MNP Brownian motion spectra.

10. The MNP heat treatment machine of claim 6 wherein the bias field has a gradient and heating is performed along a surface within the sample space.

11. The MNP heat-treatment machine of claim 10 further comprising apparatus configured to rotate a subject in the treatment space relative to the magnetic field.

12. The MNP heat-treatment machine of claim 11 wherein the apparatus configured to rotate a subject in the treatment space relative to the magnetic field comprises a subject rotator.

13. The MNP heat-treatment machine of claim 10 wherein there are a plurality of sets of magnets configured to provide a magnetic field having a strength gradient through the treatment space, and where the sets of magnets configured to provide a magnetic field having a strength gradient through the treatment space are operated in a sequence to rotate the magnetic field about the treatment space.

14. A method of imaging first magnetic nanoparticle (MNP) concentrations in a subject comprising:
applying a magnetic bias field having a gradient to the subject;
applying pulses of a radio frequency field to the subject at an electron paramagnetic resonant frequency of the first MNPs in the magnetic bias field;
sweeping a parameter selected from the group consisting of strength of the magnetic bias field strength and the radio frequency to sweep a surface of resonance through the subject;
observing electron paramagnetic resonances of the first MNPs;
rotating the magnetic bias field relative to the subject and sweeping the surface of resonance through the subject while observing additional electron paramagnetic resonances of the first MNPs; and
reconstructing first MNP concentrations in a first three-dimensional model of the subject.

15. The method of claim 14 further comprising imaging second magnetic nanoparticle (MNP) concentrations in the subject by a method comprising:
applying a magnetic bias field having a gradient to the subject;
applying pulses of a radio frequency field to the subject at an electron paramagnetic resonant frequency of the second MNPs in the magnetic bias field;
sweeping a parameter selected from the group consisting of strength of the magnetic bias field strength and the radio frequency field to sweep a surface of resonance through the subject;
observing electron paramagnetic resonances of the second MNPs;
rotating the magnetic bias field relative to the subject and sweeping the surface of resonance through the subject while observing additional electron paramagnetic resonances of the second MNPs; and
reconstructing second MNP concentrations in a second three-dimensional model of the subject.

16. The method of claim 15 further comprising subtracting the second three dimensional model of the subject from the first three-dimensional model of the subject.

17. The method of claim 14 wherein the MNPs are complexed with antibodies to a particular tissue type.

18. The method of claim 17 further comprising heating the MNPs by applying radio frequency energy at a frequency of electron paramagnetic resonances of the MNPs.

19. The method of claim 18 further comprising observing an MNP Brownian motion spectrum to determine a temperature of the MNPs.

20. The method of claim 14 further comprising heating the MNPs by applying radio frequency energy at a frequency of electron paramagnetic resonances of the MNPs.

21. The method of claim 20 further comprising observing an MNP Brownian motion spectrum to map a temperature of the MNPs within the subject.
